(19) United States
(12) Patent Application Publication
(10) Pub. No.: US 2019/0367971 A1
(43) Pub. Date: Dec. 5, 2019

(54) METHODS FOR HAPLOTYPE AND DIPLOTYPE DETERMINATION

(71) Applicant: The Hong Kong University of Science and Technology, Hong Kong (CN)

(72) Inventors: I Ming Hsing, Hong Kong (CN); Tsz Wing Fan, Hong Kong (CN); Henson Lim Lee Yu, Hong Kong (CN)

(73) Assignee: THE HONG KONG UNIVERSITY OF SCIENCE AND TECHNOLOGY, Hong Kong (CN)

(21) Appl. No.: 16/478,078

(22) PCT Filed: Mar. 14, 2018
(86) PCT No.: PCT/CN2018/078938

§ 371 Date: Jul. 15, 2019

Related U.S. Application Data
(60) Provisional application No. 62/601,136, filed on Mar. 14, 2017.

(51) Int. Cl. C12Q 1/6827

(52) U.S. Cl. CPC C12Q 1/6827; C12Q 2565/101; C12Q 2537/1376; C12Q 2563/143; C12Q 2525/186; C12Q 2563/149

(57) ABSTRACT

The present invention provides methods for determining a haplotype or a diplotype in a genetic sample. The method comprises the steps of contacting a probe-complex with the genetic sample, wherein the probe complex comprises at least two probes, hybridizing at least two probes to a polynucleotide sequence, each of which is specific to one of two or more genetic variants, determining the presence or absence of at least one genetic variant by detecting a signal emitted from at least one probe, wherein detection of said signal is indicative of the the presence of a genetic variant, removing or displacing at least one of said probes from said sample, and detecting a change in the signal to determine the haplotype or a diplotype in the genetic sample. Kits for use in the method of the invention are also provided.

### CROSS-REFERENCE TO RELATED APPLICATIONS

This application claims the benefit of priority of U.S. provisional application 62/601,136, filed on 14 Mar. 2017, the contents of it being hereby incorporated by reference in its entirety for all purposes.

### FIELD OF THE INVENTION

The invention is in the field of genetics. In particular, the field of allelic mapping, haplotyping and diplotyping.

### BACKGROUND OF THE INVENTION

Variations in the genetic sequence have been identified at multiple sites in the human genome. Genetic variations or polymorphisms may have functional implications, for example, some polymorphisms may predispose and individual to a disease or may determine the way in which drugs are metabolized. Polymorphisms exist in different forms and include single nucleotide variations, multibase insertion, microsatellite repeats, di-nucleotide repeats, tri-nucleotide repeats and sequence rearrangements. Among these sequence polymorphisms, the most frequent polymorphisms in the human genome are single-base variations, also called single-nucleotide polymorphisms (SNPs).

In the human genome, SNPs occur at approximately every three hundred bases. Since humans are diploid organisms, multiple SNPs can be inherited together and appear on one strand of DNA, or inherited from both parents separately so that they appear on different copies of the same gene. The phase information, that is, whether the SNPs occur on the same strand (cis-) or on different strands (trans-) with each other, is important as it is known to affect disease risk, severity of disease phenotype, and drug response. Some mutations can mask the deleterious effects of another when they occur cis to each other. For example, thrombophilia is associated with two mutation sites in the methylenetetrahydrofolate reductase (MTHFR) gene; a C→T mutation at position 677 (C677T) and an A→C, mutation at position 1298 (A1298C). However, it is only when these two mutations occur trans to each other will the diseased phenotype be observed. In other cases, the effects are compounded when the mutations are cis with each other such as that for two independent SNPs related to lung cancer and parkinson's disease. There is also growing evidence that around 1-5% of human genes are expressed in an allele-specific manner. This means that one copy can be expressed at a different rate than the other; thus, if the combination of SNPs affect the function of the corresponding protein, the sequence information on each allele is also important.

Despite the importance of SNP phasing, this information is often missed out in routine DNA sequencing technologies since the DNA is randomly fragmented prior to sequencing. Typically, phase information is obtained by processing the genotype data of a father-mother-child trio through computational and statistical algorithms such as PHASE and HelixTree. However, this method is limited by the accuracy of the bioinformatics software and the availability of family data. Rare variants which occur at low frequencies also cannot be phased. Alternatively, direct laboratory-based approaches may be employed. These include long-range sequencing combined with more powerful computational tools, conventional sequencing methods applied to a single molecule of DNA to detect only cis-SNPs, or by the sequential addition of dibases to resolve phase information using the neighbouring bases.

Typically, phasing of haplotypes and diplotypes requires multiple iterative rounds of polymerase chain reaction (PCR) and the products of the second (or afterwards) rounds of PCR are sequenced in order to determine which SNPs are co-expressed. Currently, the most widely used routine phasing method is Sanger sequencing, which requires specialized instrumentation and is only limited to SNPs within 700 nt.

Presently, all the methods for determining haplotypes and diplotypes are limited by labour-intensive protocols and the need for specialized instrumentation and high-powered computing devices for data analyses.

There is a need to provide a simple and quick method to resolve the exact allelic content on the chromosomal copies to determine haplotypes and diplotypes that overcomes or at least ameliorates one or more of the disadvantages described above.

### SUMMARY

In one aspect, there is provided a method for determining a haplotype or a diplotype in a genetic sample comprising the steps of: a) contacting a probe-complex with the genetic sample, wherein the probe complex comprises at least two probes, b) hybridising at least two probes to a polynucleotide sequence, wherein each of the at least two probes is specific to one of two or more genetic variants in said polynucleotide sequence; c) determining the presence or absence of at least one genetic variant by detecting a signal emitted from at least one probe, wherein detection of said signal is indicative of the the presence of a genetic variant; d) removing or displacing at least one of said probes from said sample; and e) detecting a change in the signal to determine the haplotype or a diplotype in the genetic sample.

In one aspect, there is provided a kit for use in the method as disclosed herein comprising at least two probes, wherein each of the at least two probes is specific to one of two or more genetic variants, and instructions for use.

### DEFINITIONS

As used herein, a “genetic variant” refers to a variation in one or more nucleotides in a genetic sequence relative to a reference nucleotide sequence.

As used herein, the term “haplotype” refers to two or more alleles on one chromosome or a part of a chromosome. The term “haplotype” may also refer to two or more single nucleotide polymorphisms (SNPs) on one chromosome or part of a chromosome.

As used herein, the tem “diplotype” refers to the matched pair of haplotypes on homologous chromosomes.

As used herein, the term “allele” refers to any one of two or more different forms of a gene that occupy the same position (locus) on a chromosome.

As used herein, the term “phase” or “phasing” refers to the position of one genetic variant or SNP relative to another genetic variant or SNP. Two or more genetic variants or SNPs that occur on the same nucleic acid strand are in cis-configuration whilst two or more genetic variants or SNPs that occur on different nucleic acid strands are in trans-configuration.

As used herein, the term “hybridize” or grammatical variants thereof means that that the probe anneals to a target polynucleotide sequence via a non-covalent interaction. It will be generally understood that any hybridization reaction is performed under stringent conditions. The term “stringent conditions” means any hybridisation conditions which allow the probe to bind specifically to a nucleotide sequence, but not to any other nucleotide sequences. It is within the ambit of the skilled person to vary the parameters of hybridization such as temperature, probe length and salt concentration such that specific hybridisation can be achieved.

As used herein, the term “probe” refers to a molecule designed to bind to a nucleotide sequence and may be used to identify a specific nucleotide sequence in a target or sample. The probe may comprise a nucleotide sequence complementary to the specific nucleotide sequence to be identified.

As used herein, the term “toehold” in the context of “toehold sequence” refers to a single stranded nucleic acid sequence within a probe which binds to a given target nucleic acid sequence. Binding of the toehold sequence to the target triggers separation of the strands of the probe.

As used herein, the term “polymorphism” refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs.

As used herein, the term “single nucleotide polymorphism” or “SNP” is a single base pair change in a nucleotide sequence. Typically a single nucleotide polymorphism is the substitution of one nucleotide by another nucleotide at the polymorphic site. Deletion of a single nucleotide or insertion of a single nucleotide may also give rise to single nucleotide polymorphisms. Typically, between different genomes or between different individuals, the polymorphic site may be occupied by two different nucleotides.

### BRIEF DESCRIPTION OF THE DRAWINGS

The invention will be better understood with reference to the detailed description when considered in conjunction with the non-limiting examples and the accompanying drawings, in which:

FIG. 1 shows a schematic diagram of a two-step conditional displacement hybridization assay (CDHA). FIG. 1A shows the binding of the two double-stranded probes to the corresponding SNP site when present, and a hypothetical fluorescence readout obtained for each target. FIG. 1B shows the second step of the CDHA assay which involves the addition of a polymerase. The corresponding fluorescence signals are also shown. (TD: target with two SNPs, TA: target with SNP A, TB: target with SNP B, WT: wild type).

FIG. 2 shows a schematic diagram of a conditional displacement hybridization assay (CDHA) based on a universal probe design. FIG. 2A shows the products of the X-probe and the corresponding targets with the expected time-course fluorescence signal profile for the two fluorophores. FIG. 2B shows the differentiation of TD/WT and TA/TB diplotypes upon the addition of polymerase based on the fluorescence readout. (TD: target with two SNPs, TA: target with SNP A, TB: target with SNP B, WT: wild type).

FIG. 3 shows a schematic diagram of a magnetic bead-mediated conditional separation assay for SNP phasing. FIG. 3A shows the hypothetical fluorescence signatures when a three-stranded probe attached to a magnetic bead via a streptavidin-biotin linker is reacted with the four possible targets. FIG. 3B shows the differentiation of TD/WT and a TA/TB diplotypes using magnetic bead separation. (TD: target with two SNPs, TA: target with SNP A, TB: target with SNP B, WT: wild type).

FIG. 4 shows a schematic diagram of three SNP phasing assay using a conditional displacement hybridization assay (CDHA). FIG. 4A shows the different targets with 3 SNP sites. FIG. 4B shows the overall schematics of a three-SNP phasing assay consisting of two pools of probes that will undergo the same hybridization step and conditional displacement via a polymerase. In pool 1, probe B has a 3′ modification that prevents polymerase extension, while in pool 2, probe A has the same 3′ modification. The toehold and recognition sequences for all three probes in both pools remain the same. FIG. 4C shows the fluorescence signals of a target in probe pool 1 and probe pool 2 before the addition of the polymerase. It further enumerates the haplotypes that do not have any change in fluorescence signal even after the addition of polymerase. This means that samples containing these haplotypes will only be identified in the other sample well. FIG. 4D shows the tabulation of results and that all possible combinations of diplotypes for targets with three SNP sites can be phased via conditional displacement hybridization assay.

FIG. 5 shows a schematic diagram of a three SNP phasing assay using conditional separation via magnetic beads assay. FIG. 5A shows two sample wells with different placement of the magnetic bead—probe C in sample well 1, and probe B in sample well 2. FIG. 5B shows that after magnetic separation, haplotypes containing SNP C will be retained in sample well 1, while haplotypes containing SNP B will be retained in sample well 2. The results of the fluorescence signal readout before and after magnetic separation are summarized in FIG. 5C and FIG. 5D for sample wells 1 and 2 respectively, showing that all possible combinations of diplotypes for targets with three SNP sites can be phased via the conditional separation assay.

FIG. 6 shows the results of SNP phasing in 10 combinations of diplotypes. FIG. 6A shows the fluorescence measurement of the 10 different diplotypes. Each sample containing a specific diplotype was incubated with the interrogation probes (for SNP A and SNP B) for 30 min, followed by the addition of a polymerase and dNTP and another 30 minutes of incubation. The amount and type of SNPs present on the two strands were indicated by the fluorescence signals given in the initial probe hybridization step, with the darker line corresponding to SNP A and the lighter line corresponding to SNP B. That is, a diplotype containing two, one, or no SNP will register a fluorescence signal in the range of 0.8-1, 0.5-0,7, and 0.1-0,3, respectively. This fluorescence information is able to identify at most eight diplotypes as shown in the figure but is unable to classify between the diplotypes formed by TD+WT and TA+TB (outlined in a box in the figure), which contained the same amount and type of SNPs. Whether the two SNPs are in cis (TD+WT) or Trans (TA+TB) with each other is then determined by the change in the fluorescence signal for SNP A (darker line) in the second step of the reactions. Only the cis configuration will cause a significant drop in the fluorescence signal (i.e., 0.2-0.3 au). The coloured circles below the fluorescence curves indicate the number of SNP A and B present on the two sets of strands as inferred by the fluorescence values, and are summarized in the table in FIG. 6B.

### DETAILED DESCRIPTION OF THE PRESENT INVENTION

In a first aspect the present invention refers to a method for determining a haplotype or a diplotype in a genetic sample comprising the steps of: a) contacting a probe-complex with the genetic sample, wherein the probe complex comprises at least two probes; b) hybridising at least two probes to a polynucleotide sequence, wherein each of the at least two probes is specific to one of two or more genetic variants in said polynucleotide sequence; c) determining the presence or absence of at least one genetic variant by detecting a signal emitted from at least one probe, wherein detection of said signal is indicative of the the presence of a genetic variant; d) removing or displacing at least one of said probes from said sample; and e) detecting a change in the signal to determine the haplotype or a diplotype in the genetic sample.

In one embodiment, a probe-complex may comprise at least two probes. A probe-complex may also comprise a double stranded nucleic acid molecule, for example, a double stranded DNA molecule, a three-stranded nucleic acid molecule for example, a three stranded DNA molecule or a four-stranded nucleic acid molecule, for example, a four stranded DNA molecule. The design of a probe-complex may be determined by the application of the probe-complex. For example, a probe-complex may be designed to enable simultaneous interrogation of two genetic variants or SNPs in a sample. In another example, a probe-complex may be designed to contain a universal fluorophore and quencher pair together with probes to identify specific genetic variants or SNPs.

In one embodiment, two discrete probes may be hybridized to a connector strand to form a three stranded DNA molecule.

It will generally be understood that each probe may interact with a specific target sequence on a polynucleotide sequence. The polynucleotide sequence may be isolated or purified from a genetic sample which may include but is not limited to blood, blood plasma, serum, buccal smear, amniotic fluid, prenatal tissue, sweat, nasal swab, urine, organs, tissues, fractions, and cells isolated from mammals including humans. A genetic sample may also include sections of the genetic sample including tissues (for example, sectional portions of an organ or tissue). In other embodiments, the isolated polynucleotide sequence may be amplified by methods known in the art. In a preferred embodiment, the polynucleotide sequence tray be amplified using a polymerase chain reaction (PCR) or an isothermal amplification process.

Each probe may interact with its specific target sequence sequentially or simultaneously. The polynucleotide sequence may include nucleic acids, nucleic acid fragments, plasmids and other molecules such as gene fragments and the like. The probes may be nucleic acids, oligonucleotides, nucleic acid variants such as peptide nucleic acids (PNAs) or locked nucleic acids (LNAs), peptides, proteins, dyes, fluorophores, magnetic beads, lipids, drugs, or small molecules. Any combination of probe types may be used in a given experiment.

In some embodiments, the probe may comprise one or more nucleotide sequences that are specific to one or more target sequences.

A probe may further comprise a dye. A dye may refer to a substance used to color materials or to enable the generation of luminescent or fluorescent signal. A dye may absorb or emit light at specific wavelengths and may be bound to the a probe or a target by intercalation, noncovalent binding or covalent binding. A dye may be a chemiluminescent or a fluorophore molecule. Examples of chemiluminescent molecules include but are not limited to N-(4-Aminobutyl)-N-ethylisoluminol, luminol, coelenterazine, ruthenium complexes such as Ru(BPS)₃⁴⁺ (wherein BPS is 4,7-diphenyl-1,10-phenanthroline disulfonate or bathophenanthroline disulfonate), Ru(BPS)₂(bipy)²⁻ (where bipy is 2,2′-bipyridine), Ru(BPS)(bipy)₂ and tris(2,2′-bipyridine)ruthenium(II) (Ru(bipy)₃^{2'}) and analogues of ruthenium. A fluorophore may be a protein or peptide, a small organic compound, or a synthetic oligomer or polymer. For example, a fluorophore may be a non-protein organic fluorophore selected from xanthene derivatives, cyanine derivatives, squarine derivatives and ring-substituted squaraines, napthalene derivatives, coumarin derivatives, oxadiazole derivatives, anthracene derivatives, pyrene derivatives, oxazine derivatives, arylmethine derivatives and tetrapyyrole derivatives. Other chemiluminescent or fluorophore molecules known in the art may be suitably used within the scope of the invention. Those skilled in the art will also recognise other dyes that may be used within the scope of the invention. It will also be understood to those skilled in the art that any set or combination of fluorophores or dyes may be used, but these should have non-overlapping excitation/emission spectra.

In one embodiment, a probe may further comprise a quencher. A quencher is any molecule or agent that decreases chemiluminescence or fluorescence intensity. An example of a quencher may be an organic or inorganic molecule with a network of conjugated double-bonds. Other examples of quenchers include but are not limited to molecular oxygen, iodide ions and acrylamide. In one embodiment, the fluorophore and quencher may be located on separate strands of the probe.

In the method of the present invention, the at least two probes may comprise at least two distinct fluorophores. The at least two probes may further comprise at least two quenchers which may be identical or distinct from each other.

In a further embodiment, one or more quenchers may be added onto a connector strand hybridized to at least two discrete probes. The connector strand may be covalently modified with one or more quencher molecules at the 5′ and/or 3′ end of the connector strand.

In another embodiment, one of the at least two probes may further comprise a magnetic bead. The magnetic bead may be attached to one or more of the at least two probes via a streptavidin molecule. In one embodiment, the magnetic bead may be a streptavidin-modified magnetic bead functionally attached to a probe by a biotin modification in the probe. In another embodiment, the magnetic bead may be functionally attached to a probe by activation of a functional group (e.g. N-hydroxysuccinimide (NHS)) on the surface of the magnetic bead and reacting the magnetic bead with a probe comprising one or more amine-functionalized oligonucleotides.

In some embodiments, the probe may be immobilized onto a surface. The surface may be a solid surface or a substrate. Examples of a surface include but are not limited to gold or silica, a membrane such as egg shell membrane (ESM), a polymeric substrate or a gel. In some embodiments, the probe may be attached to a solid surface via a gold-thiol-DNA bond, a silica-NHS-amine-DNA interaction or a polymer-streptavidin-biotin-DNA interaction.

Hybridization of a probe to nucleotide sequence may be achieved by any means that anneals the probe to the nucleotide sequence. In one embodiment, hybridization may be achieved by toehold-mediated strand displacement. Hybridization may be triggered by a toehold sequence on the probe annealing to a complementary sequence on the polynucleotide sequence. Annealing of the toehold sequence on the probe to the polynucleotide sequence may cause the strands of the probe to separate and the strand of the probe comprising the toehold sequence to hybridize to the polynucleotide sequence. The strand that does not comprise the toehold sequence is displaced. It will generally be understood that the specificity of the hybridization reaction of the probe to the polynucleotide sequence may be governed thermodynamically by the sequence of the probe and/or the length of the toe-hold region.

Hybridization of the probe to the polynucleotide sequence may result in the emission of a signal from the probe. Detection of the presence of an emitted signal may be indicative of the presence of a genetic variant. In other embodiments, the intensity of the emitted signal may be measured to determine the presence or number of copies of a genetic variant in a genetic sample. The intensity of the emitted signal may be measured relative to a reference signal. A reference signal may be a signal emitted from a genetic sample with known genetic variants, for example, a wild type sample. A reference signal may also be a signal emitted from the same genetic sample prior to the addition of an enzyme or prior to the removal or displacement of one or more probes. A reference signal may also be a signal emitted from a genetic sample in the absence of hybridization of a probe to the polynucleotide sequence.

As described herein, the presence or absence of a genetic variant may be determined by detecting a signal emitted from at least one probe using the method of the present invention. The method of the present invention further allows the phase of at least two or more genetic variants to be determined by removing or displacing at least one of said probes from the sample.

In one embodiment, the at least one probe may be removed by magnetic separation. It will be understood that magnetic separation may be used to separate a probe attached to a magnetic bead from a probe that is not attached to a magnetic bead.

In another embodiment, the at least one probe may be removed from the sample by a washing step. In one embodiment, the at least one probe may be immobilized to a surface and after the target nucleic acid hybridizes with the immobilized probe, any probes that are not immobilized to the surface after the hybridization step may be removed from the sample by washing.

In another embodiment, the at least one probe may be displaced from said sample by the action of a polymerase. In some embodiments, a probe that is bound to the polynucleotide sequence may act as a primer for the polymerase. Extension of the primer by the polymerase enzyme may displace another probe that is bound to the polynucleotide sequence.

In a preferred embodiment, the polymerase may be a high fidelity DNA polymerase. The high fidelity DNA polymerase may have no 5′ to 3′ exonuclease activity.

In one embodiment, one of the at least two probes further comprises a modification that prevents polymerase extension. An example of a modification that prevents polymerase extension is an overhanging region. For example, an overhanging region may comprise a 3′ poly A tail. An overhanging region may also comprise a hairpin region. In a preferred embodiment, one of the at least two probes may be modified with a 3′ poly A tail.

Removal or displacement of the at least one probe from the genetic sample may result in a change in the presence or level of intensity of one or more signals emitted from the genetic sample. Detection of a change in one or more signals emitted may be used to determine the haplotype or diplotype in the genetic sample. In one example, detection of a decrease in an emitted signal may indicate that two genetic variants are located in cis configuration. In another example, detection of a decrease in an emitted signal may indicate that two genetic variants are located in trans configuration.

The method of the present invention may be used to determine the haplotype or diplotype of two or more genetic variants. The two or more genetic variants may be located less than 1 kilobase (1 kb) from each other, or more than 1 kb from each other. It will generally be understood that the two or more genetic variants may be located at a distance of up to the length of a chromosome apart. For example, the two or more genetic variants may be located at least 100 nucleotides (nt), at least 200 nt, at least 300 nt, at least 400 nt, at least 500 nt, at least 600 nt, at least 700 nt, at least 800 nt, at least 900 nt, at least 1000 nt, at least 1500 nt or at least 2000 nt from each other. In one embodiment, the two or more genetic variants are located on a chromosome. In a preferred embodiment, the two or more genetic variants may be located at least 700 nt apart.

The present invention also provides a probe-complex for use in the method of the present invention, comprising at least two probes, a fluorophore and a quencher.

In one embodiment, the probe-complex may further comprise a connector strand hybridized to the at least two probes. In another embodiment, the probe-complex further comprises a magnetic bead attached to one or more of the at least two probes.

The present invention also provides a kit for use in the method as disclosed herein comprising at least two probes wherein each of the at least two probes is specific to one of two or more genetic variants, and instructions for use.

In one embodiment, the kit may further comprise a polymerase enzyme.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

### Example 1

### Conditional Displacement by Polymerase

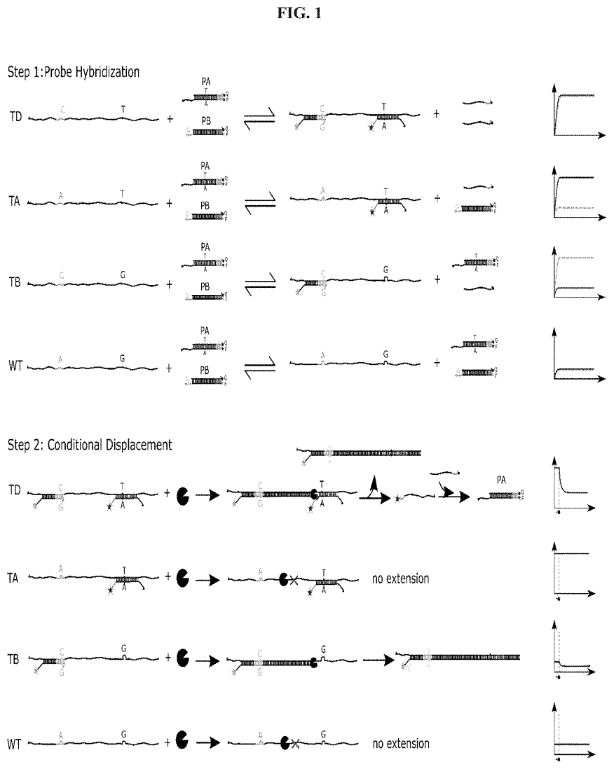

A two-step reaction was designed wherein the presence of the two SNPs was first interrogated, and if both SNPs were present, the phase information was deduced from a second set of nucleic acid reactions (see FIG. 1). A synthetic target DNA was designed to contain one, both, or none of the two possible SNP sites labeled A and B, which are 100 nt apart. Subsequently, two double stranded DNA (dsDNA) probes containing a fluorophore-quencher pair were added, each probe interrogating one of the SNP sites in the target DNA. If the SNP was present, that segment of DNA hybridized with the fluorophore-containing strand of the first probe and displaced the second quencher-containing strand through a toehold-mediated strand displacement reaction. The specificity of this reaction was governed thermodynamically by the sequence of the probe as well as the length of the toehold region. Similarly, a second probe containing a different fluorophore was used to interrogate the presence of a second SNP. In solution, the generation of both fluorescence signals cannot differentiate cis- from trans-SNPs. Thus, a high fidelity DNA polymerase with no 5′→3′ exonuclease activity was added. The binding of the first probe acted as a primer and was extended by the polymerase, and consequently displaced the second probe downstream to the first. This reduced the corresponding fluorescence indicating a cis-SNP configuration. This did not occur when the SNPs were found in different strands.

### Example 2

### Conditional Displacement by Polymerase Using a Universal Probe

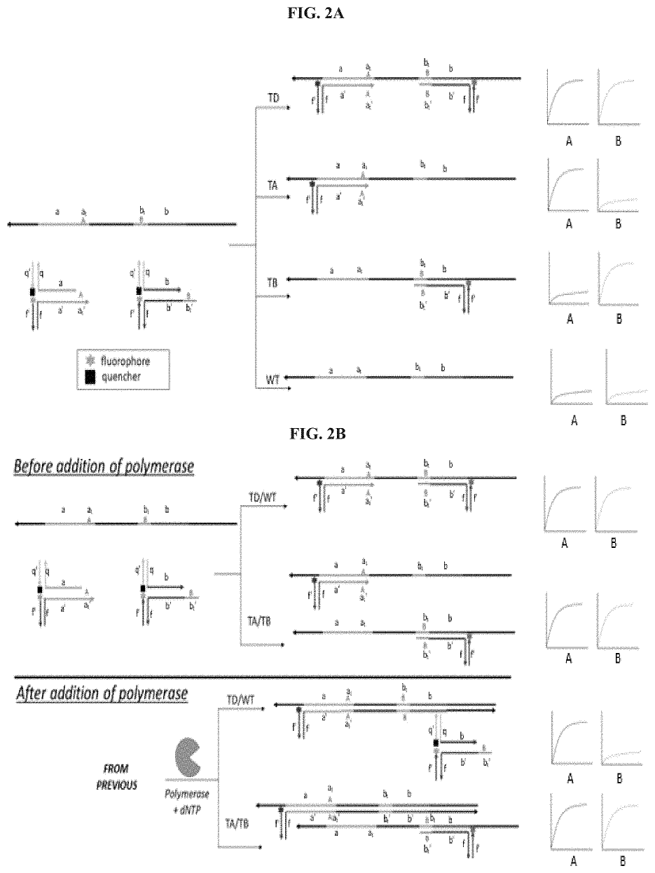

A modification of the previous example was introduced by using a universal probe, (henceforth referred to as “X-probe” because of its shape as shown in FIG. 2). The probe was first prepared by hybridizing four strands of DNA—a fluorophore-labelled strand, a quencher-labelled strand, and two sequence-specific strands. To phase multiple pairs of SNPS, the same fluorophore-labelled and quencher-labelled strands were used, but the sequence-specific strands were changed accordingly. The resulting X-probes were then used to phase multiple pairs of SNPs in one assay using a 96-well plate by loading different X-probes in each well and performing the same protocol as the previous section (FIG. 2). Since the fluorophore-quencher pairs are similar, the same two fluorescence channels were used. This method yielded a higher throughput and a more efficient method of phasing two or more pairs of SNPs simultaneously.

### Example 3

### Conditional Separation by Magnetic Separation

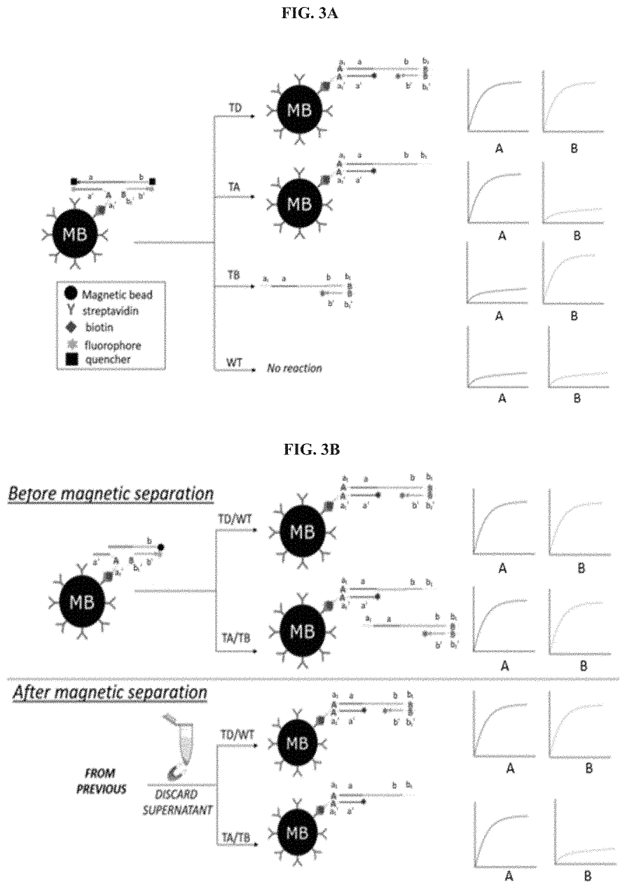

The present invention also provides a method where an enzyme is not needed. Instead, two fluorophore-labelled probes that interrogate the two different SNP sites (referred to as SNPs A and B) were initially hybridized together with a connector strand that was covalently modified with quencher molecules on both the 5′ and the 3′ end. This three-stranded probe was then attached to a streptavidin-modified magnetic bead via a biotin modification in one of the fluorophore-labelled probe. The fluorescence signal was measured twice—first after incubation of the magnetic bead-conjugated probe with the appropriate target DNA and a second time after separation, washing, and reconstitution (to the same volume) of the magnetic bead. In the first measurement, no signal was registered without the target (or in the presence of a wild type target) and one fluorophore was used to detect when the target contains either of the SNPs. Both fluorophores emitted a signal when both are present (FIG. 3). Up to this step, the fluorescence signal profile yielded ambiguous signals in the presence of the diplotypes TD/WT and TA/TB. Given that only one fluorophore-labelled strand is attached to a magnetic bead, only one fluorescence signal was left when each SNP was found in different strands upon separation by a magnet, while both signals remained when both SNPs were found in a single strand (FIG. 3).

### Example 4

### Phasing of Three SNPs by Conditional Displacement by a Polymerase

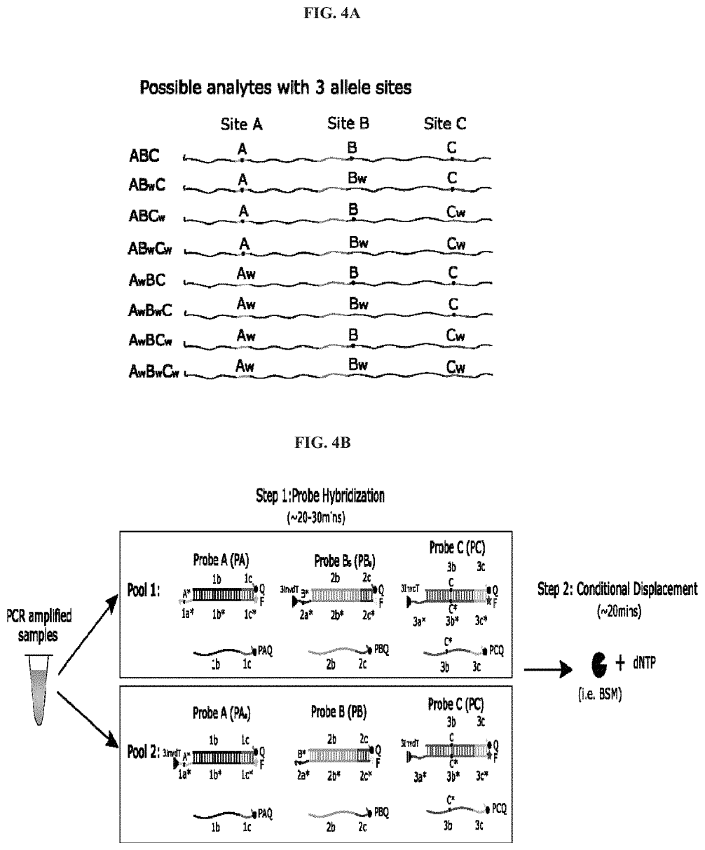

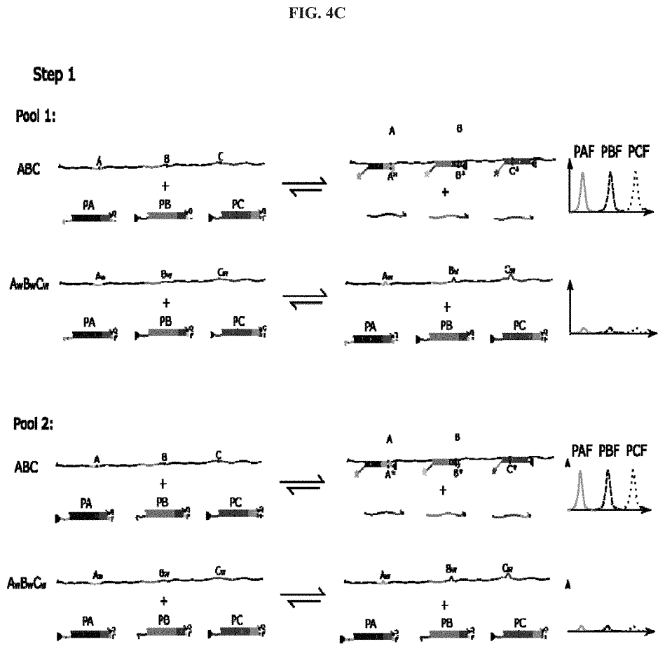

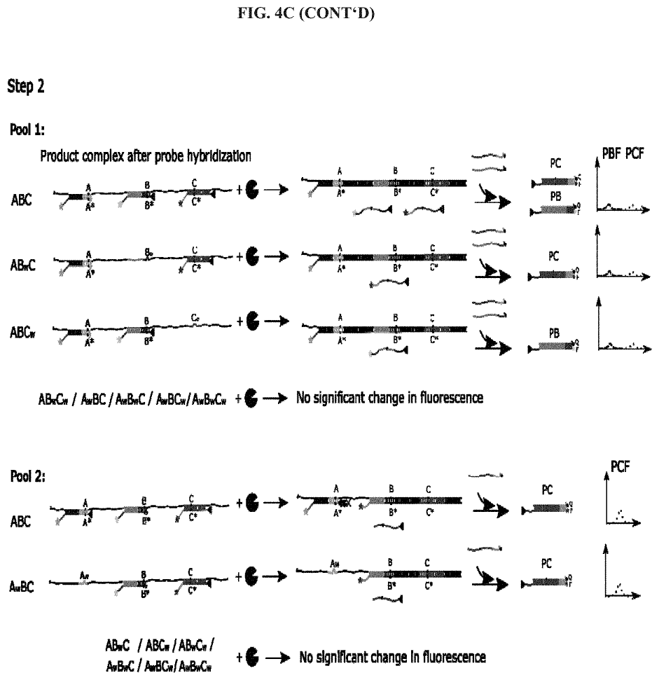

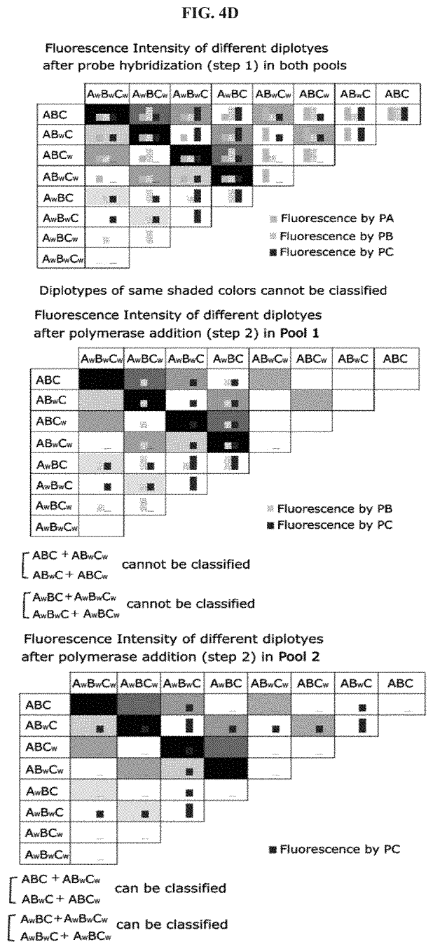

The method in Example 1 previously described was extrapolated to phase three SNP sites. Phasing multiple SNP sites increases the repertoire of the diseases and conditions that can be identified and expands the possible applications of this technology. In this case, two reaction vessels containing the same sample (containing any one, two, or three SNPs) were incubated with three fluorophore-quencher pairs (probe A, B, and C) to interrogate the presence of the three SNPs, SNPs A, B, and C, respectively. The wavelengths of the fluorescence signals emitted were indicative of the presence of the corresponding SNP (FIG. 4). In order to deduce the phase information, a slightly modified probe A and probe B was used in each reaction vessel. In the first sample well, the probe B used was appended with a 3′ poly-A tail so that upon the addition of the polymerase, only probe A can be extended. Similarly, in the second sample well, the probe A used was appended with a 3′ poly-A tail (FIG. 4B). Thus, upon the addition of the polymerase, the reduction in fluorescence signals in the first sample well provided the phase information of SNP B and C with respect to SNP A and the reduction in the fluorescence signal in the second sample well provided the phase information of SNP C with respect to SNP B (FIG. 4C). As certain diplotypes are only discernible in one of the two sample wells; that is in one sample well, there are more than one diplotypes that can yield the same fluorescence signal after the reaction takes place, it is necessary to run two different reactions. By running two different reactions (in sample well 1 and sample well 2), the method ensures that even if two (or more) diplotypes have the same fluorescence signature in sample well 1, they will have different fluorescence signature in sample well 2. Thus, both sample wells are required to ascertain that all diplotypes can be differentiated. Using the combined information of both sample wells, the phase information for the three SNPs was resolved. (FIG. 4D).

### Example 5

### Phasing of Three SNPs by Conditional Separation by a Magnetic Particle

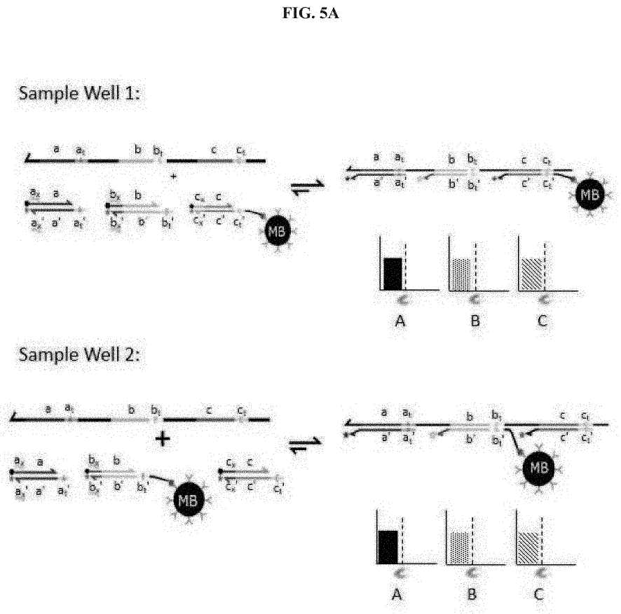

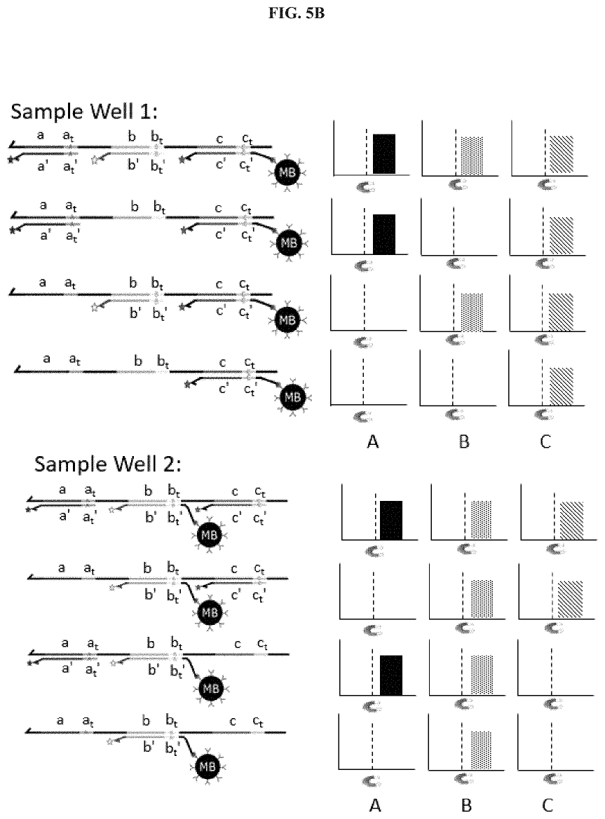

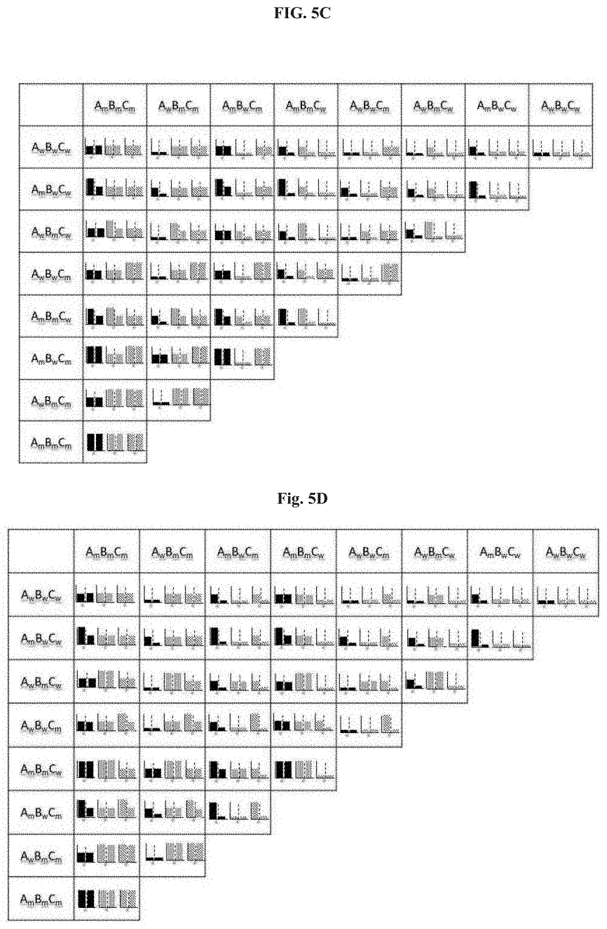

Similar to Example 4, the conditional separation by a magnetic particle (Example 3) was extrapolated to interrogate and phase three SNPs. This required two reaction vessels wherein the magnetic bead was attached to probe C in the first sample well, and attached to probe B in the second sample well (FIG. 5).

In the first sample well, the fluorescence profile after magnetic separation provided the phase information relative to probe C, while the second sample well provided the phase information relative to probe B (FIG. 5B). As in Example 4, running two different reactions (in sample well 1 and sample well 2) ensures that even if two (or more) diplotypes have the same fluorescence signature in sample well 1, they will have different fluorescence signature in sample well 2. Using the combined information from both sample wells, the phase information for the three SNPs was resolved (FIG. 5C and 5D).

### Example 6

### Phasing of 10 Combinations of Diplotypes

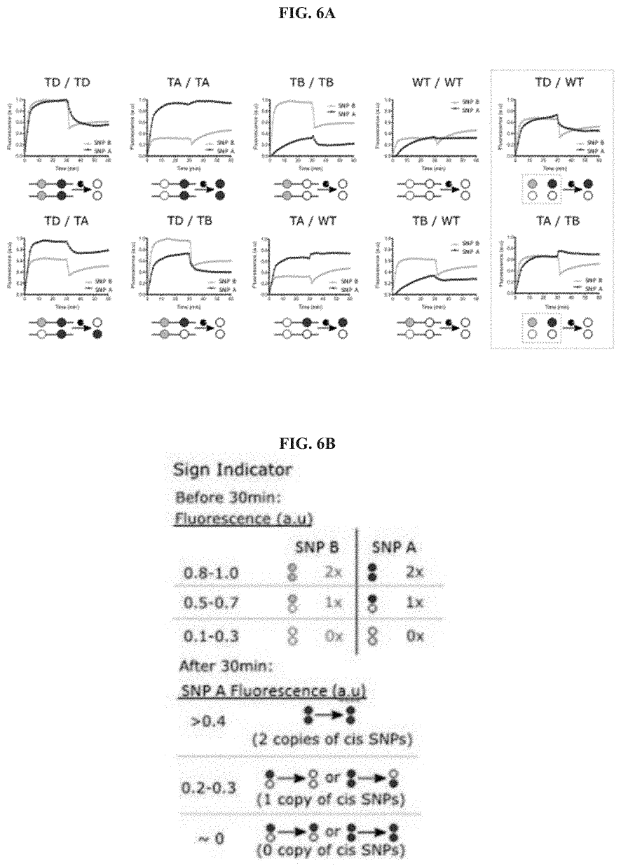

The conditional displacement assay was tested on 10 possible diplotypes using the four templates (TD, TA, TB and WT) as shown in FIG. 6. By symbolizing them as full, half and no record (i.e. two, one, or no coloured circles) based on their fluorescence values upon saturation at both stages, all 10 diplotypes presented unique barcodes according to their number and phase of SNPs. Before enzyme addition, both green and red fluorescence values quantitatively correlated to the number of SNPs A and B present. For instance, green fluorescence of ˜1 for the case when there are 2 TD strands, 2 TB strands, or a combination of TD and TB—all of which contained two SNP B. This was symbolically denoted as two green circles. A fluorescence value of ˜0.6 was observed for a combination of TD and TA, TD and WT, TB and TA or TB and WT—all of which contained one SNP B—and were represented by one green circle. Finally a fluorescence signal of ˜0.2 was observed for those without SNP B (i.e., 2 TA strands, 2 WT strands, or a combination of TA and WT), which were denoted by two hollow green circles. These notations applied similarly to the red fluorescence channel. Using this notation, the mixtures of TA and TB from TD and WT, which both registered a half record for red and green signal cannot yet be distinguished. This was determined by the second part of the assay when the polymerase was added. Red fluorescence remained almost constant when TA and TB were present but dropped by one third for that of TD and WT. Fluorescence reduction for other SNP combinations was also consistent.

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A method for determining a haplotype or a diplotype in a genetic sample comprising the steps of:
a) contacting a probe-complex with the genetic sample, wherein the probe complex comprises at least two probes;
b) hybridising the at least two probes to a polynucleotide sequence, wherein each of the at least two probes is specific to one of two or more genetic variants in said polynucleotide sequence;
c) determining the presence or absence of at least one genetic variant by detecting a signal emitted from at least one probe, wherein detection of said signal is indicative of the the presence of a genetic variant;
d) removing or displacing at least one of said probes from said sample; and
e) detecting a change in the signal to determine the haplotype or a diplotype in the genetic sample.

2. The method of claim 1, wherein the probe-complex comprises a double-stranded DNA (dsDNA) molecule, a three-stranded DNA molecule or a four-stranded DNA molecule.

3. The method of claim 2, wherein the probe-complex comprises a three stranded DNA molecule comprising a first and second probe hybridised to a connector strand, wherein the first and second probes are discrete from each other.

4. The method of claim 1, wherein each of the at least two probes comprises a different fluorophore.

5. The method of claim 4, wherein each of the at least two probes further comprises a quencher.

6. The method of claim 5, wherein the fluorophore and quencher are located on separate strands of the probe.

7. The method of claim 6, wherein the quenchers are located on the connector strand.

8. The method of claim 1, wherein one of the at least two probes further comprises a magnetic bead.

9. The method of claim 8, wherein the magnetic bead is a streptavidin-modified magnetic bead functionally attached to the said one probe by a biotin modification in the probe.

10. The method of claim 1, wherein the probe is immobilized onto a solid surface.

11. The method of claim 10, wherein the solid surface is gold, silica, or a polymeric substrate.

12. The method of claim 1, wherein the at least one probe is hybridized by toehold-mediated strand displacement.

13. The method of claim 1, wherein the at least one probe is removed from the sample by magnetic separation.

14. The method of claim 1, wherein the at least one probe is displaced by the action of a polymerase.

15. The method of claim 14, wherein polymerase is a high fidelity DNA polymerase with no 5′ to 3′ exonuclease activity.

16. The method of claim 1, wherein one of said at least two probes further comprises a modification that prevents polymerase extension.

17. The method of claim 1, wherein detection of the signal or change in the signal further comprises detecting the intensity of the signal or change in the intensity of the signal relative to a reference signal to determine the haplotype or diplotype in the genetic sample.

18. The method of claim 17, wherein the reference signal is the signal detected from a known haplotype or diplotype.

19. The method of claim 1, wherein the presence or absence of the two or more genetic variants is determined simultaneously.

20. A probe-complex for use in the method of claim 1, comprising at least two probes, a fluorophore and a quencher.

21. The probe-complex of claim 20, further comprising a connector strand hybridized to the at least two probes.

22. The probe-complex of claim 21, further comprising a magnetic bead attached to one or more of the at least two probes.

23. A kit for use in the method of claim 1, comprising at least two probes, wherein each of the at least two probes is specific to one of two or more genetic variants, and instructions for use.

24. The kit of claim 23, further comprising a polymerase enzyme.
